(19) United States
(12) Patent Application Publication
(10) Pub. No.: US 2022/0047598 A1
(43) Pub. Date: Feb. 17, 2022

(54) Use of Casein Kinase 1 Inhibitors For Treating Vascular Diseases

(71) Applicant: Qanatpharma AG, Stanz (CH)

(72) Inventor: Steffen-Sebastian Bolz, Herrsching (DE)

(21) Appl. No.: 17/274,773

(22) PCT Filed: Sep. 19, 2019
(86) PCT No.: PCT/EP2019/074012

§ 371 Date: Mar. 9, 2021

(30) Foreign Application Priority Data
Sep. 9, 2018 (EP) 18193320.1

(51) Int. Cl. A61K 31/519; A61K 31/506; A61P 9/10

(52) U.S. Cl. CPC A61K 31/519; A61P 9/10; A61K 31/506

(57) ABSTRACT

The present invention relates to the use of casein kinase 1 inhibitors for treating vascular diseases, preferably peripheral vascular diseases, and to corresponding treatment methods.

### FIELD OF THE INVENTION

The present invention relates to the use of casein kinase 1 inhibitors for treating vascular diseases, preferably peripheral vascular and cardiovascular diseases. The present invention also relates to corresponding treatment methods.

### BACKGROUND OF THE INVENTION

Despite significant investment into basic and clinical cardiovascular research, cardiovascular disease remains the most devastating and challenging health issue worldwide: it is responsible for over 17,500,000 deaths each year (equaling 47% of all deaths caused by non-communicable diseases). The global costs for managing cardiovascular diseases will rise to $1.04 trillion by 2030, thereby transforming this health issue into a serious threat to the global economy.

Hypertension is the #1 risk factor for all cardiovascular diseases: it increases the risk of heart attack, heart failure (HF), stroke and kidney failure. As many as 1 in 3 people have high blood pressure. However, since hypertension has few signs or symptoms, most cases remain undiagnosed. We do not understand why people develop hypertension: only 10% of hypertension cases can be explained. Understanding hypertension is key to preventing and treating cardiovascular disease.

Microvascular research studies the structure and function of the smallest blood vessels (pre-capillary arterioles and post-capillary venules) in health and disease. Resistance arteries are “hotspots” within the cardiovascular system that regulate both mean arterial pressure (MAP) and tissue perfusion, and are responsible for generating the largest portion of the total peripheral resistance (TPR). Consequently, changes to their structure and function (e.g. through diseases or aging) immediately affect tissue perfusion and MAP. Resistance arteries are an untapped opportunity for improving cardiovascular health. Understanding the structural and molecular basis of microvascular function in health and disease will unlock a range of new therapeutic strategies.

Current treatments that target the microvasculature are sub-standard as they fail to improve organ function and are difficult to titrate to effective doses without incurring significant side-effects. As an example, augmented myogenic tone in HF elevates TPR and induces an “afterload mismatch” that induces deleterious long-term effects on ventricular topography and cardiac function. In fact, even a short-term augmentation in afterload induces significant infarct expansion. Consequently, several strategies that reduce cardiac afterload in HF have been evaluated, however, with limited success. While reducing cardiac afterload may confer cardiac benefits, vasodilators (e.g., nitric oxide) bear the risk of eliminating myogenic responsiveness and dangerously lowering TPR and blood pressure.

Myogenic responsiveness and impacts on systemic hemodynamics: In 1902, Sir William Bayliss discovered that transmural pressure elevation causes resistance arteries to “writhe like a worm” (i.e., constrict)<1>. This observation, later termed the myogenic response<2> <5>, describes this dynamic adjustment of blood vessel diameter to changes in perfusion pressure. As the etymology of its name implies (myo=muscle, genic=generated), the myogenic response originates from the vascular wall smooth muscle layer<6 8>; anatomically, the myogenic response is a property of pre-capillary small arteries and arterioles (i.e., small diameter “resistance arteries” are functionally distinct from large diameter conduit arteries).

On the systemic level, Ohm's law indicates that resistance artery myogenic reactivity will have significant impact on TPR and MAP<9,10>. This positions resistance arteries as a “functional hotspot” in several disease processes hallmarked by changes in tissue perfusion and/or systemic hemodynamics, including cardiomyopathy<11>, HF<12,13>, diabetes<14,15> and hypertension<16>. In vivo, skeletal muscle resistance arteries prominently modulate TPR, as this vascular bed forms the body's largest circulatory network<17>.

TNF reverse signaling as a regulator of myogenic responsiveness: In our seminal study investigating skeletal muscle resistance arteries<18>, we demonstrated that tumour necrosis factor (TNF), and more specifically the membrane-bound version (mTNF), is a constitutive mechanosensor driving the myogenic response in skeletal muscle resistance arteries under a non-pathological setting. Accordingly, acutely deleting the TNF gene in smooth muscle cells or scavenging TNF with Etanercept attenuates skeletal muscle resistance artery myogenic responsiveness and hence, reduces systemic blood pressure. Notably, Etanercept's inhibitory effect on skeletal muscle artery myogenic responsiveness is conserved across five distinct species, including humans. Mechanistically, mTNF transduces the mechanical load imposed on vascular smooth muscle cells into an outside-in signal (i.e., a “reverse signal” through TNF) that connects to the established intracellular myogenic signaling elements (e.g., ERK 1/2 and sphingosine kinase 1). This non-canonical mTNF reverse signaling mechanism appears to be unique to skeletal muscle resistance arteries<18>.

Casein Kinase 1 is a Modulator of TNF Reverse Signaling: TNF's cytoplasmic domain does not possess discernible enzymatic function and hence, signals through associated proteins including casein kinase 1 (CK1). Remarkably, evolutionary pressure has conserved TNF's CK1 phosphorylation site across several species, suggesting that it serves a critical function<19>. In this regard, TNF phosphorylation acts as an “activity switch” and provides a flexible mechanism to modulate TNF reverse signaling functions.

CK1 is a family of 7 ubiquitously expressed monomeric serine/threonine protein kinases<20>. While all CK1 isoforms possess a highly conserved kinase domain, they differ significantly in their non-catalytic N- and C-terminal domains<21,22>: these domains play crucial roles in regulating kinase activity, kinase localization and substrate specificity in vivo<21 23>. Since CK1 family isoforms display similar substrate specificity in vitro<24>, their distinct biological functions in vivo (e.g., chromosome segregation, spindle formation, circadian rhythms, nuclear import, Wnt pathway signaling and cell survival/apoptosis) arise almost entirely from differences in localization, docking sequences and interaction partners<22>.

All CK1 isoforms are constitutively active and therefore, are classified as “second messenger-independent kinases”. Canonically, CK1 phosphorylates a “primed” (pre-phosphorylated) consensus sequence S(P)-X-X-S, where “S(P)” represents the “primed” phosphoserine, “X” represents any amino acid and “S” represents the target serine that CK1 phosphorylates<20>. Since efficient substrate recognition requires a phosphorylated serine residue, CK1 frequently phosphorylates substrates in conjunction with other kinases<20>, an aspect consistent with a hierarchical phosphorylation mechanism<25>.

### SUMMARY OF THE INVENTION

The technical problem underlying the present invention is to provide a novel regimen for improving myogenic responsiveness in the peripheral vascular system.

According to the invention the term “improving myogenic responsiveness in the peripheral vascular system” is meant an improvement of the myogenic responsiveness especially in a disease state or condition, respectively, in which the myogenic responsiveness is deteriorated, in particular in the context of the specific diseases and conditions, respectively, as outlined in more detail below. Compared to a healthy subject, the myogenic responsiveness in the peripheral vascular system of subjects showing a deteriorated myogenic responsiveness in the peripheral vascular system, said myogenic responsiveness is normalized or at least changed in the direction of a normalized value.

The solution to the above technical problem is provided by the embodiments of the present invention as disclosed in the claims, the present description and the accompanying figures.

The inventors identified superior means to reduce TPR by selectively targeting mechanisms that modulate discrete portions of myogenic reactivity. The vascular bed-specific modulation of myogenic responsiveness improves organ blood flow and function at full preservation of normal physiological regulatory mechanisms.

According to the present invention, compounds that alter vascular smooth muscle CK1 Expression activity have impact on mTNF reverse signaling and myogenic responsiveness, and as a consequence, total peripheral resistance, tissue blood flow, and systemic blood pressure. Since altered myogenic reactivity is a hallmark of numerous diseases (e.g., heart failure, subarachnoid haemorrhage, diabetes, stroke, sepsis), targeting microvascular CK1 activity/expression has the potential to improve microvascular myogenic responsiveness and systemic hemodynamics in diverse diseases.

More specifically, the present invention provides the use of casein kinase 1 (CK1) inhibitors, i.e. one or more CK1 inhibitors may be used, for the prevention and/or treatment of vascular and/or cardiovascular diseases (CVDs) such as coronary artery diseases (CAD) (angina and myocardia infarction (commonly known as a heart attack)), stroke, heart failure, hypertensive heart disease, rheumatic heart disease, cardiomyopathy, abnormal heart rhythms, congenital heart disease, valvular heart disease, carditis, aortic aneurysms, peripheral artery disease, thromboembolic disease, venous thrombosis, subarachnoid haemorrhage and hypertension, whereby heart failure, subarachnoid haemorrhage and hypertension are particularly preferred.

According to the invention, a CK1 inhibitor is a compound that reduces the expression and/or activity of CK1.

Preferred CK1 inhibitors for use in the invention are inhibitors selective for CK1 isoforms δ (CK1δ, in other instances also referred to as“CK1D”) and/or ε (CK1ε, in other instances also referred to as“CK1E”). In certain embodiments of the invention, it is preferred when the CK1 inhibitor has a stronger inhibitory effect on CK1δ than on CK1ε a Particularly suitable CK1 inhibitors for use in the invention are CK1 inhibitors disclosed in WO 2014/023271, more preferably the CK1 inhibitors D4476, PF670462, IC261 and PF 4800567. Highly preferred CK1 inhibitors for use in the invention are PF-670462 and PF-4800567, which may also be used in combination. Other useful CK1 inhibitors in the context of the invention are CK1 δ selective inhibitors disclosed in Salado et al. (2014 J. Med. Chem. 2014, 57, 2755-2772, especially those shown in FIG. 1, Table 1 and FIG. 2 thereof, with the compound M3-15 being most preferred. It is to be understood that the present invention is also directed to the use of pharmaceutically acceptable salts, solvates, esters salts of such esters, as well as any other adduct or derivative which upon administration to a patient in need is capable of providing, directly or indirectly, a CK1 inhibitor for use in the invention or a metabolite or residue thereof.

According to the invention, CK1 inhibitors inhibit CK1 Expression/activity and reduce mTNF reverse signaling in the vascular smooth muscle cells of peripheral resistance arteries, in particular in patients suffering from diseases like vascular and cardiovascular diseases (CVDs) such as coronary artery diseases (CAD) (angina and myocardia infarction (commonly known as a heart attack)), stroke, heart failure, hypertensive heart disease, rheumatic heart disease, cardiomyopathy, abnormal heart rhythms, congenital heart disease, valvular heart disease, carditis, aortic aneurysms, peripheral artery disease, thromboembolic disease, venous thrombosis, subarachnoid haemorrhage and hypertension, whereby heart failure, subarachnoid haemorrhage and hypertension are particularly preferred.

According to the invention, CK1 inhibitors reduce smooth muscle Erk1/2 phosphorylation and sphingosine-1-phosphate signaling in diseases like heart failure, in particular in patients suffering from diseases like vascular and cardiovascular diseases (CVDs) such as coronary artery diseases (CAD) (angina and myocardia infarction (commonly known as a heart attack)), stroke, heart failure, hypertensive heart disease, rheumatic heart disease, cardiomyopathy, abnormal heart rhythms, congenital heart disease, valvular heart disease, carditis, aortic aneurysms, peripheral artery disease, thromboembolic disease, venous thrombosis, subarachnoid haemorrhage and hypertension, whereby heart failure, subarachnoid haemorrhage and hypertension are particularly preferred.

According to the invention, CK1 inhibitors inhibit CK1 Expression/activity and reduce peripheral myogenic reactivity, in particular in patients suffering from diseases like vascular and cardiovascular diseases (CVDs) such as coronary artery diseases (CAD) (angina and myocardia infarction (commonly known as a heart attack)), stroke, heart failure, hypertensive heart disease, rheumatic heart disease, cardiomyopathy, abnormal heart rhythms, congenital heart disease, valvular heart disease, carditis, aortic aneurysms, peripheral artery disease, thromboembolic disease, venous thrombosis, subarachnoid haemorrhage and hypertension, whereby heart failure, subarachnoid haemorrhage and hypertension are particularly preferred. According to the invention, CK1 inhibitors reduce total peripheral resistance, in particular in patients suffering from diseases like vascular and cardiovascular diseases (CVDs) such as coronary artery diseases (CAD) (angina and myocardia infarction (commonly known as a heart attack)), stroke, heart failure, hypertensive heart disease, rheumatic heart disease, cardiomyopathy, abnormal heart rhythms, congenital heart disease, valvular heart disease, carditis, aortic aneurysms, peripheral artery disease, thromboembolic disease, venous thrombosis, subarachnoid haemorrhage and hypertension, whereby heart failure, subarachnoid haemorrhage and hypertension are particularly preferred.

According to the invention, CK1 inhibitors reduce systemic blood pressure, in particular in patients suffering from diseases like vascular and cardiovascular diseases (CVDs) such as coronary artery diseases (CAD) (angina and myocardia infarction (commonly known as a heart attack)), stroke, heart failure, hypertensive heart disease, rheumatic heart disease, cardiomyopathy, abnormal heart rhythms, congenital heart disease, valvular heart disease, carditis, aortic aneurysms, peripheral artery disease, thromboembolic disease, venous thrombosis, subarachnoid haemorrhage and hypertension, whereby heart failure, subarachnoid haemorrhage and hypertension are particularly preferred.

According to the invention, CK1 inhibitors increase blood flow in skeletal muscle, mesenteric, renal, and coronary circulations, in particular in patients suffering from diseases like vascular and cardiovascular diseases (CVDs) such as coronary artery diseases (CAD) (angina and myocardia infarction (commonly known as a heart attack)), stroke, heart failure, hypertensive heart disease, rheumatic heart disease, cardiomyopathy, abnormal heart rhythms, congenital heart disease, valvular heart disease, carditis, aortic aneurysms, peripheral artery disease, thromboembolic disease, venous thrombosis, subarachnoid haemorrhage and hypertension, whereby heart failure, subarachnoid haemorrhage and hypertension are particularly preferred.

According to the invention, the effect of CK1 inhibitors is limited to myogenic tone; catecholamine-induced vasoconstriction is not affected in diseases like heart failure, subarachnoid haemorrhage, and hypertension.

According to the invention, the effect of CK1 inhibitors is limited to the peripheral and coronary circulation; cerebrovascular hemodynamics is not affected in diseases such as those as described above, in particular in heart failure, subarachnoid haemorrhage, and hypertension. The present invention is also directed to a CK1 inhibitor, preferably a CK1 inhibitor selected from those as outlined in more detail above, for use in the prevention and/or treatment of vascular and/or cardiovascular diseases as described above.

The present invention is furthermore directed to a method for preventing and/or treating of a vascular disease and/or cardiovascular disease as described above comprising administering an effective amount of at least one CK1 inhibitor, preferably at least one CK1 inhibitor selected from those as outlined in more detail above, to a patient, preferably a mammalian patient, in particular a human patient in need thereof.

In the present invention, it is shown that the effect of CK16/e-selective inhibition plateaus and thus, it is concluded that maximal CK1 inhibition is capable of only partially attenuating myogenic vasoconstriction. In response to this comment, we conducted an additional experiment to confirm CK1 inhibition efficacy in the heart failure (HF) setting as an exemplified condition amenable to the inventive treatment using CK1 inhibitors.

According to the invention one or more CK1 inhibitor(s) can be used in its/their free form. In other embodiments the CK1 inhibitor(s) (i.e. at least one CK1 inhibitor) as used in the present invention is present in a pharmaceutical composition comprising said at least one CK1 inhibitor typically in combination with at least one pharmaceutically acceptable excipient, diluent, carrier and/or vehicle.

The effective amount of the CK1 inhibitor to be applied in the method and uses of the invention, i.e. the specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

Preferred doses in the context of the invention, in particular with reference to the preferred compounds as referred to herein, more specifically PF-4800567 and PF-670462, are about 1 to about 300 mg per kg body weight (hereinafter referred to as “mg/kg”), preferably about 10 to about 100 mg/kg, more preferably about 20 to about 70 mg/kg, particularly preferred about 25 to about 45 mg/kg such as 30 mg/kg. The dose may be administered once or more often, such as twice or thrice daily. Preferably, doses are administered once or twice daily.

Preferred administration of the one or more CK1 inhibitor(s) takes place once daily.

It is known that many cardiovascular incidents regularly take place during the resting state, i.e. at night when human patients are concerned. Therefore, the at least one CK1 inhibitor is preferably administered according to a regime that provides for a maximal pharmacological effect of the at least one CK1 During the resting state of the treated subject, more preferably at or around mid rest phase, whereby “around mid rest phase” is preferably from about −2 to about +2 hours of mid rest phase, more preferably from about −1 to about +1 hour of mid rest phase, even more preferred from about −0.5 to about +0.5 hours of mid rest phase. It is clear for the person skilled in the art that the regime providing maximal pharmacological effect of the at least one CK1 inhibitor depends on the chosen CK1 inhibitor. For example, a CK1 inhibitor displaying comparatively fast degradation of the active compound may be administered once daily at a suitable time before rest phase (i.e. before bed time for human patients) such as 3, 4, 5, or 6 hours before rest phase of the patient. In alternative embodiments where the chosen CK1 inhibitor is a compound displaying slow degradation the CK1 inhibitor can be administered through a controlled release composition ensuring that the inhibitor has maximal effect, typically maximal bioavailable concentration in the treated patient during the rest phase, preferably at or around mid rest phase (preferably according to the definition given above). Appropriate pharmaceutical compositions and/or dose and administration regimens employing, e.g. the specific CK1 inhibitors as outlined in more detail herein, for providing the above-described maximal effect during the resting phase are known to the skilled person.

The administration route for the CK1 inhibitor is not particularly critical and the chosen route depends on the individual CK1 inhibitor compound or compounds applied and the subject to be treated. Preferably the CK1 inhibitor(s) is/are administered systemically such as orally or by i.v. administration, which oral administration particularly preferred.

The terms “patient” and “subject” are used herein interchangeably and each mean an animal, preferably a mammal, and most preferably a human.

The at least one CK1 inhibitor, preferably in the present of a pharmaceutical composition as outlined above, for use according to the invention may be administered using any amount and any route of administration effective for treating the cerebrovascular condition. It is understood according to the invention that the term “treating” or “treatment” means that the severity of the condition does at least not progress as compared to the non-treated condition, preferably the severity of the condition does not progress, more preferably, the severity of the condition is lessened, even more preferred substantially lessened, and, ideally, the condition is cured to a substantial extent. Preferably, the severity of the condition according to the invention is reduced at least by 30%, more preferably by at least 50%, particularly by at least 70%, even more preferred by at least 90%, with complete cure of the condition being the most preferred outcome of the inventive treatment.

### BRIEF DESCRIPTION OF THE DRAWINGS

The Figures show:

FIG. 1: (A) Prior to treatment, myogenic tone in cremaster arteries isolated from mice with HF (n=6) is augmented relative to arteries isolated from sham control (n=8). (B) Following treatment with 550 nM PF670462 in vitro (30 minutes), myogenic tone in both groups is attenuated to same level. Thus, the HF-mediated augmentation of myogenic tone is eliminated (i.e., the post-treatment curves overlap) and the new tone level is only modestly reduced compared to the sham.

FIG. 2: shows that cytosolic portion of mTNF regulates reverse signaling.

FIG. 3: shows that casein kinase 1 regulates mTNF-mediated myogenic responsiveness.

FIG. 4: shows that casein kinase 1 delta regulates myogenic responsiveness.

FIG. 5: shows that CK1D regulates circadian oscillations in myogenic responsiveness.

FIG. 6: shows that CK1D inhibition improves microvascular dysfunction and cardiac performance in HF

The present invention is further illustrated by the following non-limiting examples.

### DETAILED DESCRIPTION OF THE INVENTION

### Examples

### Example 1: CK1 Inhibition Reduces Myogenic Responsiveness In Vitro and In Vivo

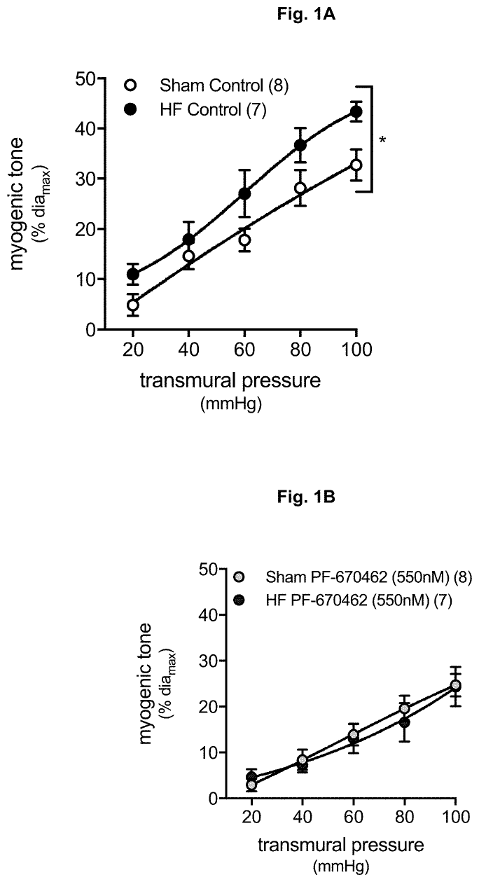

Referring to FIG. 1, microvascular smooth muscle cells were cultured form mouse mesenteric arteries and ERK1/2 phosphorylation was assessed by standard western blotting. Murine cremaster skeletal muscle resistance arteries were assessed by pressure myography. mTNF reverse signaling was induced by the intrinsically active TNF type I receptor construct, sTNFR1-Fc.

In microvascular smooth muscle cells, sTNFR1-Fc increases phosphorylation of ERK1/2. sTNFR1-Fc-induced ERK1/2 phosphorylation is abolished by both the pan CK1 inhibitor CKI-7 and the specific CK16 inhibitor PF-670462. These findings were verified in cremaster skeletal muscle resistance arteries. In vitro application of CKI-7 abrogates sTNFR1-Fc-induced mTNF reverse signaling and reduces myogenic responsiveness. CK1 inhibition did not affect myogenic responsiveness in TNF<−/−> arteries. In vitro, PF-670462 also reduces myogenic responsiveness and when applied in vivo, PF-67062 reduced myogenic responsiveness in isolated cremaster arteries. Importantly, none of the above treatments affected agonist-induced vasoconstriction.

### Example 2: Cytosolic Portion of mTNF Regulates Reverse Signaling

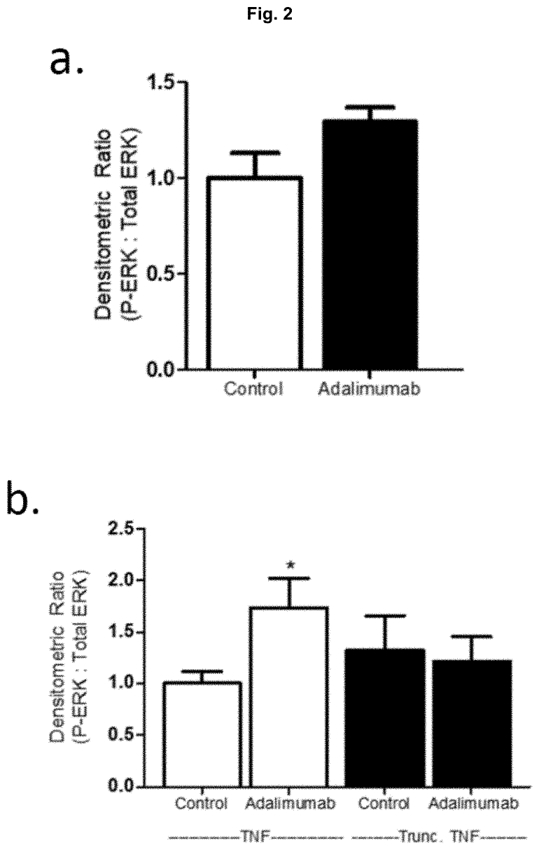

Referring to FIG. 2, human embryonic kidney (HEK) cells were transiently transfected for 24 hrs and stimulated with the TNF antibody Adalimumab (Humira™) for 5 min. mTNF reverse signaling is indicated by ERK1/2 phosphorylation, assessed by western blot. mTNF reverse signaling is not stimulated unless the HEK cells express a tumour necrosis factor (TNF) plasmid construct (NT=non-transfected); truncation of TNF's intracellular domain (amino acids 2-25) abolishes reverse signaling (Trunc TNF). Importantly, the cytoplasmic tail of mTNF contains the casein kinase 1 (CK1) recognition site (S(P)-X-X-S) that is removed in Trunc TNF, indicating the necessity for CK1 binding in mTNF reverse signaling.

<*> in FIG. 2 indicates P<0.05 by unpaired Student's t-test, n=6-12 biological replicates.

### Example 3: Casein Kinase 1 Regulates mTNF-Mediated Myogenic Responsiveness

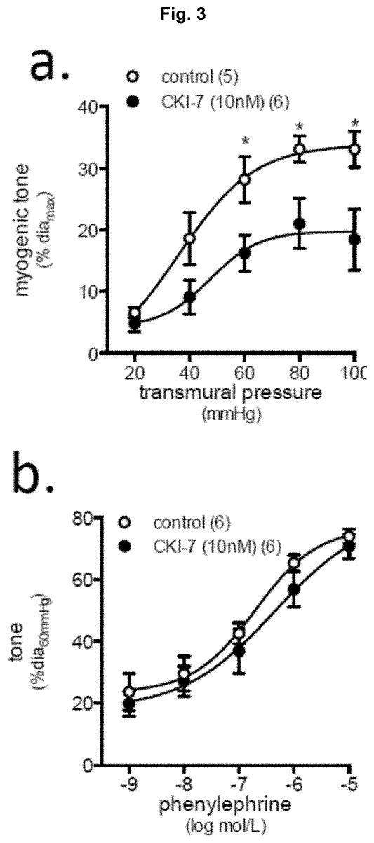

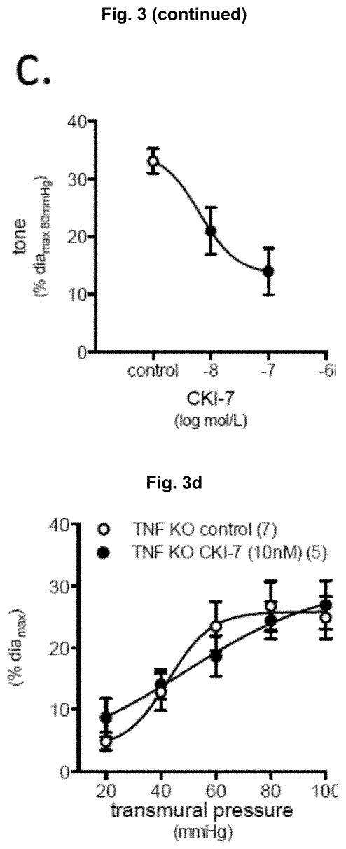

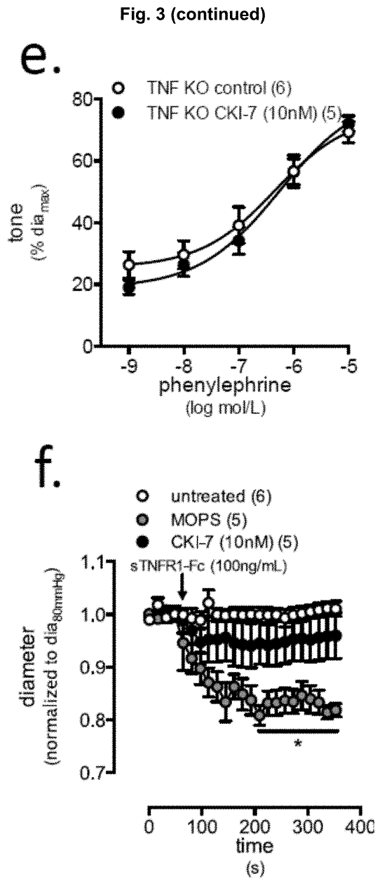

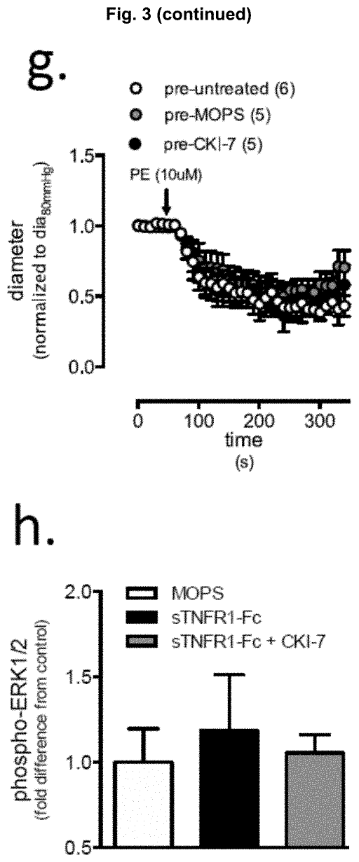

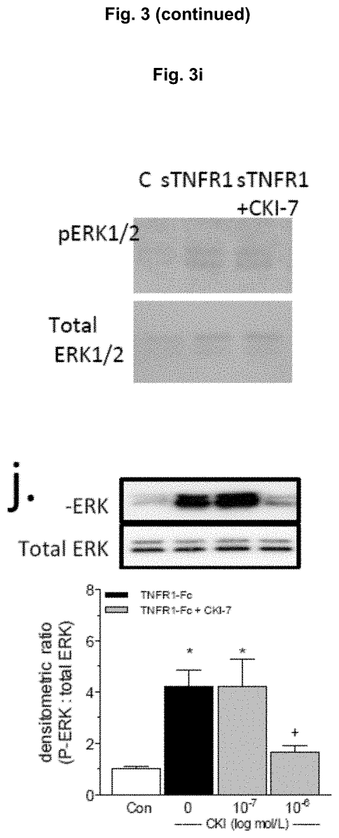

Referring to FIG. 3, mouse cremaster skeletal muscle resistance arteries were isolated and cannulated for pressure myography. Stepwise increases in transmural pressure (20-100 mmHg in 20 mmHg steps) elicit myogenic vasoconstriction. (FIG. 3a) The pan-casein kinase 1 (CK1) inhibitor CKI-7 (10 μM in vitro) reduces myogenic responsiveness. (FIG. 3b) Vessel health is not compromised by CKI-7 as phenylephrine-induced vasoconstriction remains intact. (FIG. 3c) CKI-7 elicits dose-dependent reductions in myogenic responsiveness (n=4-6 vessels). (FIG. 3d) CKI-7 is not effective in the absence of TNF signaling (i.e., TNF knockout arteries, TNF KO). (FIG. 3e) Phenylephrine-induced vasoconstriction in TNF KO arteries is not affected by CKI-7. (FIG. 3f) Acute administration of the mTNF-stimulating fusion protein sTNFR1-Fc (100 ng/mL) stimulates vasoconstriction that is prevented by CKI-7, indicating that mTNF reverse signaling requires functional CK1. (FIG. 3g) All vessels are healthy prior to experimentation, shown by robust vasoconstriction to phenylephrine (PE) (h) Isolated cremaster resistance arteries underwent pressure myography and subsequent western blotting for ERK1/2 phosphorylation, a molecular readout of mTNF reverse signaling. sTNFR1-Fc stimulates increased ERK1/2 phosphorylation that is inhibited by CKI-7. (i) Representative western blot (j) Mesenteric vascular smooth muscle cells show robust ERK1/2 phosphorylation in response to sTNFRI-Fc that is inhibited by CKI-7 (1 μM).

(FIG. 3a,b,d,e)<*> in FIGS. 3a, b, c, d and e indicates P<0.05 by unpaired Student's t-test. (FIG. 3c,f-g)<*> in FIGS. 3c, f and g indicates P<0.05 by one-way ANOVA and compared to untreated control responses by Dunnett's post-hoc test. <*> indicates P<0.05 by one-way ANOVA and compared to control (Con) and + indicates P<0.05 compared to TNFR1-Fc alone (0 mol/L CKI-7) by Bonferroni post-hoc test. The numbers of biological replicates are indicated in parentheses in FIG. 3.

### Example 4: Casein Kinase 1 Delta Regulates Myogenic Responsiveness

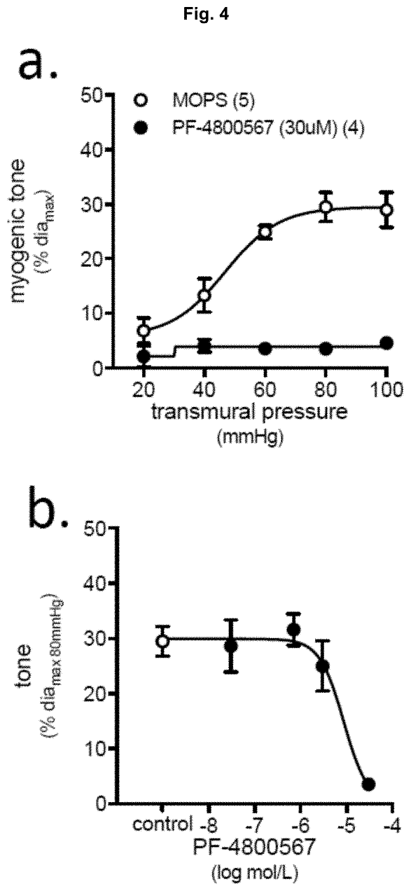

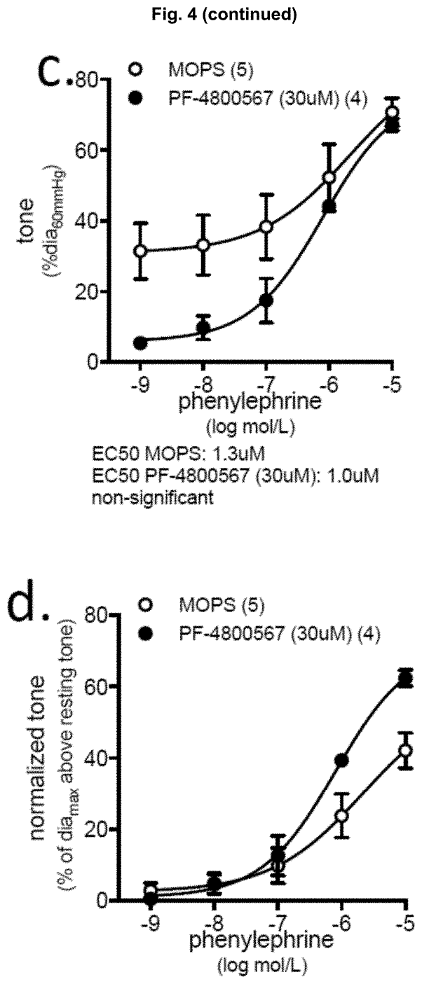

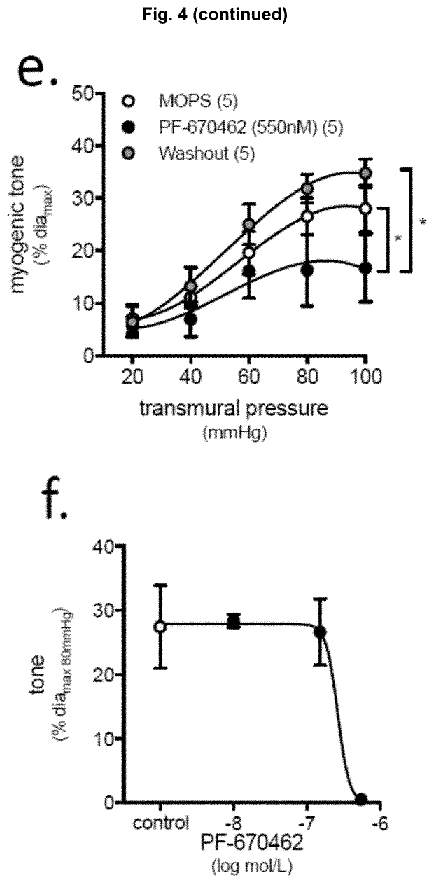

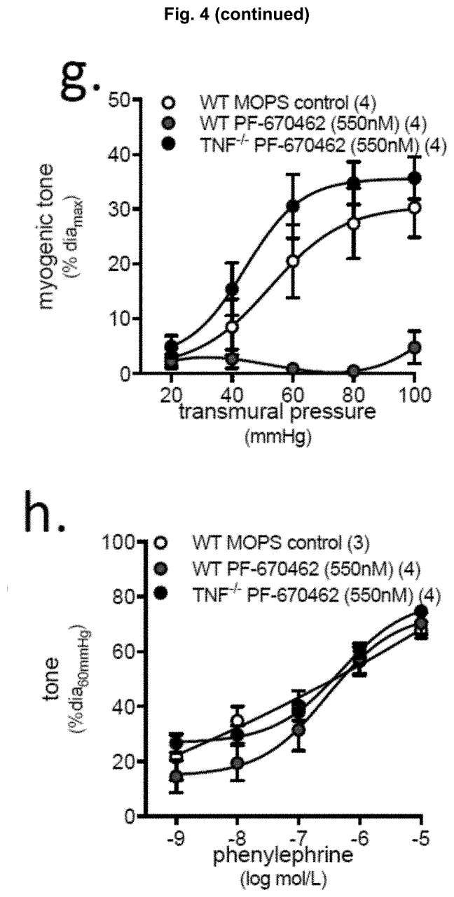

Referring to FIG. 4, mouse cremaster skeletal muscle resistance arteries were isolated and cannulated for pressure myography. Stepwise increases in transmural pressure (20-100 mmHg in 20 mmHg steps) elicit myogenic vasoconstriction. (FIG. 4a) The selective CK1E inhibitor PF-4800567 abrogates myogenic responsiveness (30 μM in vitro). (FIG. 4b) PF-4800567 elicits a dose-dependent reduction in myogenic responsiveness (n=4-5 at each dose), albeit at concentrations well above the IC₅₀ (32 nM), suggesting that this reduction in tone may be due to off-target effects of the inhibitor. (FIG. 4c) Phenylephrine-induced vasoconstriction is reduced in arteries treated with PF-4800567. (FIG. 4d) The reduction in phenylephrine-induced vasoconstriction remains after correction to baseline tone, indicating a non-specific inhibitory effect of PF-4800567 at the 30 μM dose. Together, these data indicate that CK1E is not likely mediating myogenic responsiveness, since inhibition of responses occurs only at concentrations of PF-4800567 ˜1000× greater than the IC₅₀, and PF-4800567 inhibits general vessel contractility (as indicated by blunted responses to phenylephrine). (FIG. 4e) The CK1D/E inhibitor PF-670462 reduces myogenic responsiveness (550 nM in vitro), an effect that is reversible through repeated replacements of the vessel's incubating buffer. (FIG. 4f) PF-670462 elicits dose-dependent decreases in myogenic responsiveness at concentrations approximating the published IC₅₀ (7.7-14 nM), suggesting that the reduction in tone is likely specific to either CK1D or CK1E (n=4-5 at each dose). Since the CK1E-selective inhibitor PF-4800567 was only effective well beyond the range of specificity for CK1E, we suggest that the more potent inhibitory effect of PF-670462 is due to targeting of CK1D. (FIG. 4g) CK1D signals through TNF as PF-670462 inhibits myogenic responsiveness in wild-type (WT) arteries but not in arteries from TNF knockout (TNF</>) mice. (FIG. 4h) Vessel health is not compromised by PF-670462 as phenylephrine-induced vasoconstriction is intact.

(FIG. 4a, c, d)<*> in FIG. 4a, c, and d indicates P<0.05 by unpaired Student's t-test. (FIG. 4b, f)<*> in FIGS. 4b and f indicates P<0.05 by one-way ANOVA and compared to 0 dose by Dunnett's post-hoc test. (FIG. 4e)<*> in FIG. 4e indicates P<0.05 by one-way ANOVA and compared to PF-670462 by Dunnett's post-hoc test. (FIG. 4g,h)<*> in FIGS. 4g and h indicates P<0.05 by one-way ANOVA and compared to WT PF-670462 response by Dunnett's post-hoc test. The numbers of biological replicates are indicated in parentheses in FIG. 4.

### Example 5: CK1D Regulates Circadian Oscillations in Myogenic Responsiveness

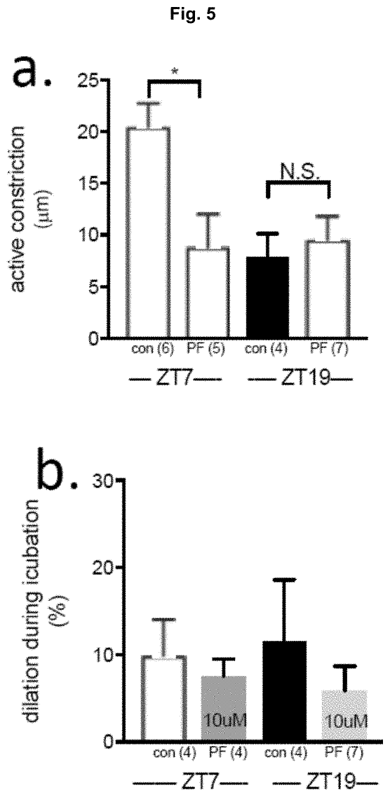

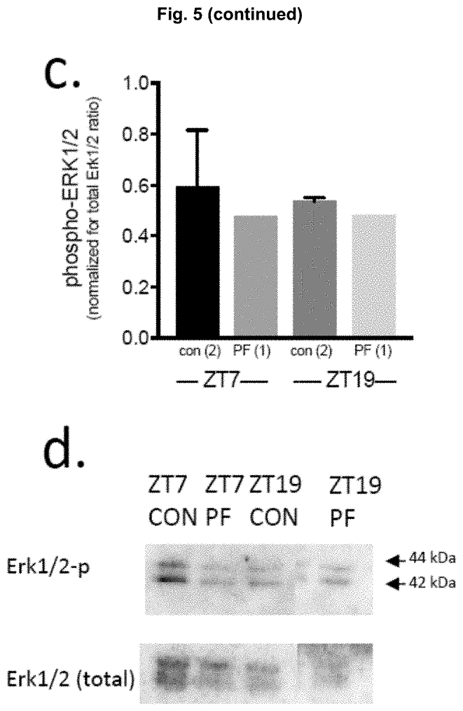

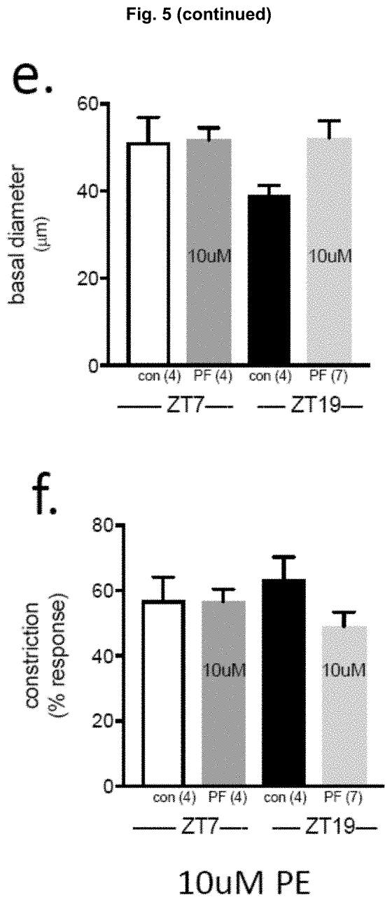

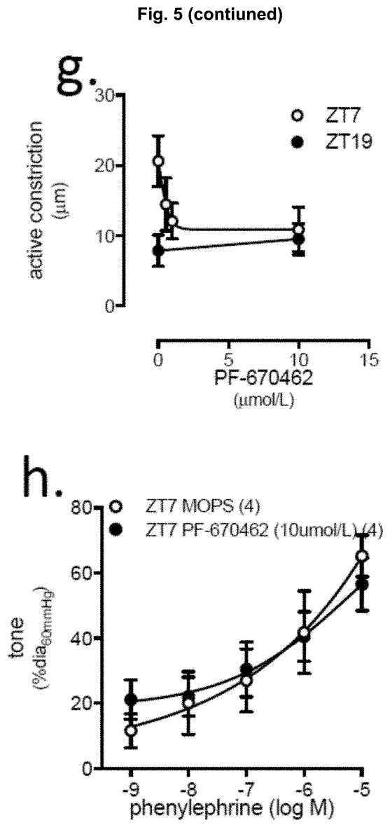

Referring to FIG. 5, mouse cremaster skeletal muscle resistance arteries were isolated at mid-rest phase (ZT7) or mid-active phase (ZT19) and cannulated for pressure myography. One-step increases in transmural pressure (60 to 100 mmHg) elicit myogenic vasoconstriction. (FIG. 5a) PF-670462 (10 μM, in vitro) reduces myogenic responsiveness only during the rest phase (ZT7) and not during the active phase (ZT19). (FIG. 5b) Vessel dilation is not affected by PF-670462 treatment, indicating that vessel initial tone is consistent prior to pressure stimulation. (FIG. 5c) Cremaster muscle resistance arteries underwent pressure myography and western blotting subsequently assessed ERK1/2 phosphorylation. PF-670462 reduces ERK1/2 phosphorylation to a greater extent during the rest phase (ZT7). (FIG. 5d) Representative western blot. (FIG. 5e) Resting diameter of the vessels is not affected by PF-670462. (FIG. 5f) Prior to administration of PF-670462, vessel health is intact as shown by robust vasoconstriction to phenylephrine. (FIG. 5g) PF-670462 dose-dependently reduces myogenic tone in the mid-rest phase (ZT7) to reach the nadir achieved in the mid-active phase (ZT19) (n=4-7 vessels per dose). (FIG. 5h) Vessel health, indicated by robust phenylephrine-induced vasoconstriction, is not affected by PF-670462 administration.

(FIG. 5a to d)<*> in FIG. 5a, b, c, and d indicates P<0.05 by unpaired Student's t-test within the same time period (ZT7 or ZT19, respectively). (FIG. 5e,f)<*> in FIGS. 5e and f indicates P<0.05 by one-way ANOVA. (FIG. 5g)<*> in FIG. 5g indicates P<0.05 by one-way ANOVA and compared to no drug (0 pmol/L) within the same time period (ZT7 or ZT 19, respectively) by Dunnett's post-hoc test. (FIG. 5h)<*> in FIG. 5h indicates P<0.05 by unpaired Student's t-test. The numbers of biological replicates are indicated in parentheses in FIG. 5.

### Example 6: CK1D Inhibition Improves Microvascular Dysfunction and Cardiac Performance in HF

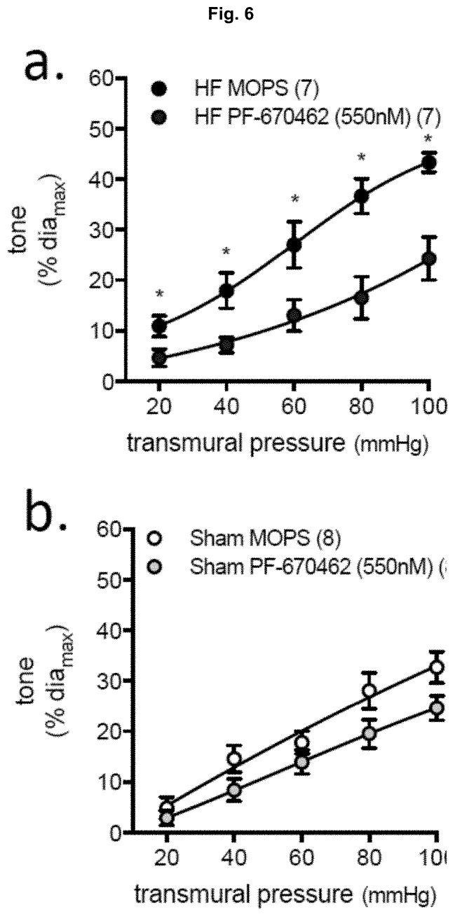

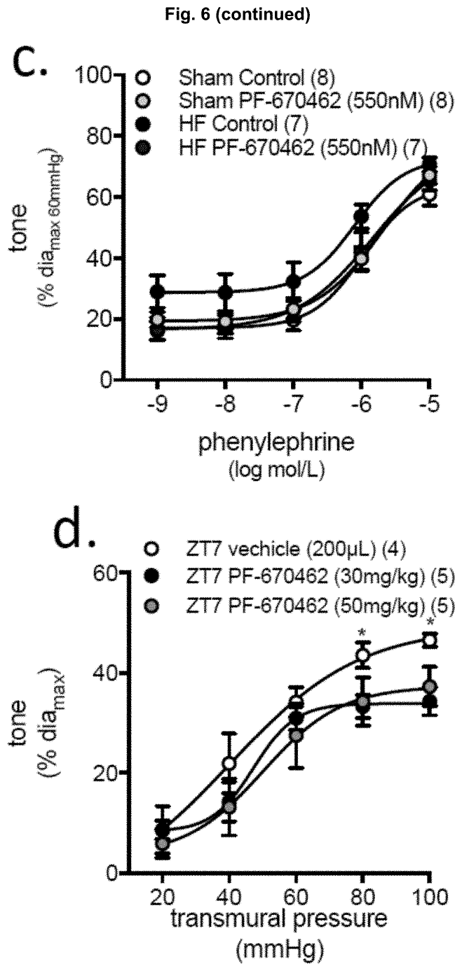

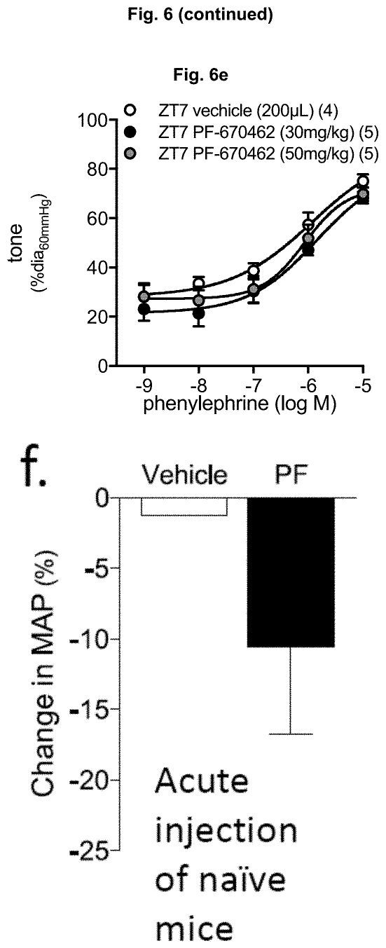

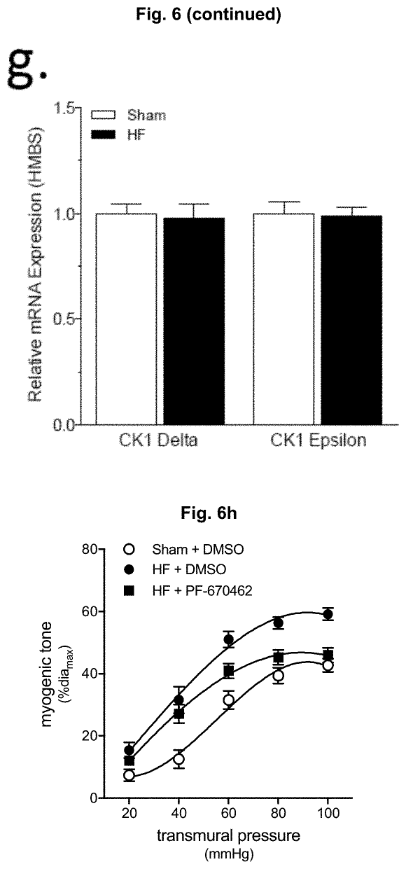

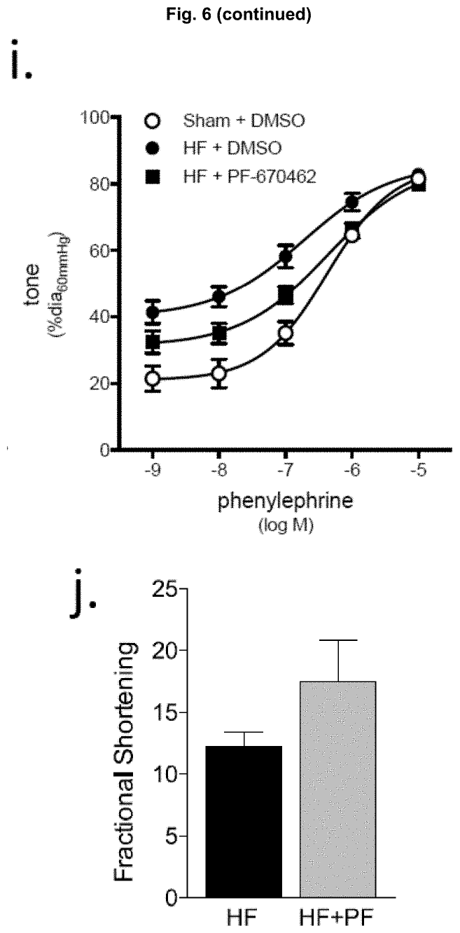

Referring to FIG. 6a to c, mice underwent myocardial infarction (ligation of the left-anterior descending coronary artery) or sham surgery. At 8 weeks post myocardial infarction, mice had developed heart failure (HF). Cremaster skeletal muscle resistance arteries were isolated and cannulated for pressure myography. Stepwise increases in transmural pressure (20-100 mmHg in 20 mmHg steps) elicited myogenic vasoconstriction that was significantly stronger in the HF than in the sham group. (FIG. 6a) In bath treatment with PF-670462 (550 nM in vitro) normalizes myogenic responsiveness in arteries from HF mice (i.e., the level of myogenic responsiveness is similar to sham values). (FIG. 6b) PF-670462 does not affect myogenic tone in sham-operated mice. (FIG. 6c) Vessels demonstrate robust function, as phenylephrine-induced vasoconstriction is intact.

Referring to FIG. 6d to f, naive mice were treated with PF-670462 (30-50 mg/kg dissolved in 200 μL water, intraperitoneal injection) or vehicle (200 μL water). 24 hrs later, in mid-rest phase (ZT7), cremaster skeletal muscle resistance arteries underwent pressure myography. (FIG. 6d) Both 30 mg/kg and 50 mg/kg of PF-670462 reduce myogenic responsiveness, suggesting that the drug is functional in vivo. (FIG. 6e) Phenylephrine-induced vasoconstriction is not altered by in vivo application of PF-670462. (FIG. 6f) In accordance with the reduction in myogenic tone, acute injection of PF-670462 (30 mg/kg intraperitoneal) reduces mean arterial blood pressure (MAP) (n=3 per group).

Referring to FIG. 6g to j, following myocardial infarction or sham surgery, mice were chronically treated with PF-670462 (30 mg/kg, intraperitoneal injection) or vehicle (DMSO) for 7 wks (5 days/wk). Cremaster skeletal muscle resistance arteries were isolated for pressure myography (FIG. 6g) mRNA expression of CK1D and CK1E in cremaster resistance arteries are not different in HF and sham (n=18 samples per group), suggesting that differences in myogenic tone are driven by post-translational mechanisms. (FIG. 6h) HF-induced elevations in myogenic tone are normalized by chronic treatment with PF-670462. (FIG. 6i)

Phenylephrine-induced vasoconstriction is intact with chronic PF-670462 treatment. Reduced tone at lower concentrations of phenylephrine in the PF-670462-treated group is the result of changes in resting myogenic tone. (FIG. 6j) Cardiac output, the quantification of blood outflow from the heart assessed with echocardiography, is elevated with chronic PF-670462 treatment.

(FIG. 6a, b, and g to j)<*> in FIGS. 6a, b and g to j indicates P<0.05 by unpaired Student's t-test. (FIG. 6c to e)<*> in FIG. 6c to e indicates P<0.05 by one-way ANOVA and compared to control by Dunnett's post-hoc test. The numbers of biological replicates are indicated in parentheses in FIG. 6.

The present invention shows that CK1 acts as a regulator of mTNF reverse signaling and hence, myogenic responsiveness. The demonstrated ability of CK1 inhibitors to reduce myogenic responsiveness without affecting agonist-induced vasoconstriction provides a substantial safety margin for clinical applications to diseases where microvascular tone is increased.

### REFERENCES

1. Bayliss. On the local reactions of the arterial wall to changes of internal pressure. J Physiol. 1902; 28:220-231.
2. Hill et ai. Invited review: arteriolar smooth muscle mechanotransduction: Ca(2+) signaling pathways underlying myogenic reactivity. J Appl Physiol (1985). 2001; 91:973-983.
3. Schubert et al. The emerging role of Ca2+ sensitivity regulation in promoting myogenic vasoconstriction. Cardiovasc Res. 2008; 77:8-18.
4. Cole et ai. Role of myosin light chain kinase and myosin light chain phosphatase in the resistance arterial myogenic response to intravascular pressure. Arch Biochem Biophys. 2011; 510:160-173.
5. Lidington et al. Capitalizing on diversity: an integrative approach towards the multiplicity of cellular mechanisms underlying myogenic responsiveness. Cardiovasc Res. 2013; 97:404-412.
6. Kuo et al. Coronary arteriolar myogenic response is independent of endothelium. Circ Res. 1990; 66:860-866.
7. Falcone et al. Endothelial independence of myogenic response in isolated skeletal muscle arterioles. Am J Physiol. 1991; 260:H130-H135.
8. Meininger et al. Cellular mechanisms involved in the vascular myogenic response. Am J Physiol. 1992; 263: H647-H659.
9. Metting et al. Systemic vascular autoregulation amplifies pressor responses to vasoconstrictor agents. Am J Physiol. 1989; 256: R98-105.
10. Metting et al. Quantitative contribution of systemic vascular autoregulation in acute hypertension in conscious dogs. J Clin Invest. 1989; 84:1900-1905.
11. Petersen et al. Coronary artery myogenic response in a genetic model of hypertrophic cardiomyopathy. Am J Physiol Heart Circ Physiol. 2002; 283:H2244-H2249.
12. Hoefer et al. Sphingosine-1-phosphate-dependent activation of p38 MAPK maintains elevated peripheral resistance in heart failure through increased myogenic vasoconstriction. Circ Res. 2010; 107:923-933.
13. Gschwend et al. Myogenic constriction is increased in mesenteric resistance arteries from rats with chronic heart failure: instantaneous counteraction by acute AT1 receptor blockade. Br J Pharmacol. 2003; 139:1317.
14. Sauve et al. Tumor Necrosis Factor/Sphingosine-1-Phosphate Signaling Augments Resistance Artery Myogenic Tone in Diabetes. Diabetes. 2016; 65:1916-1928.
15. Ungvari et al. Increased myogenic tone in skeletal muscle arterioles of diabetic rats. Possible role of increased activity of smooth muscle Ca2+ channels and protein kinase C. Cardiovasc Res. 1999; 43:1018-1028.
16. Matrougui et al. High sodium intake decreases pressure-induced (myogenic) tone and flow-induced dilation in resistance arteries from hypertensive rats. Hypertension. 1998; 32:176-179.
17. Rowell. Human cardiovascular adjustments to exercise and thermal stress. Physiol Rev. 1974; 54:75-159.
18. Kroetsch et al. Constitutive smooth muscle tumour necrosis factor regulates microvascular myogenic responsiveness and systemic blood pressure. Nat Commun. 2017; 8:14805.
19. Watts et al. A casein kinase I motif present in the cytoplasmic domain of members of the tumour necrosis factor ligand family is implicated in reverse signaling. EMBO J. 1999; 18:2119-2126.
20. Venerando et al. Casein kinase: the triple meaning of a misnomer. Biochem J. 2014; 460:141-156.
21. Gross et al. Casein kinase I: spatial organization and positioning of a multifunctional protein kinase family. Cell Signal. 1998; 10:699-711.
22. Knippschild et al. The casein kinase 1 family: participation in multiple cellular processes in eukaryotes. Cell Signal. 2005; 17:675-689.
23. Cheong et al. Casein kinase 1: Complexity in the family. Int J Biochem Cell Biol. 2011; 43:465-469.
24. Pulgar et al. Optimal sequences for non-phosphate-directed phosphorylation by protein kinase CK1 (casein kinase-1)—a re-evaluation. Eur J Biochem. 1999; 260:520-526.
25. Roach. Multisite and hierarchal protein phosphorylation. J Biol Chem. 1991; 266:14139-14142.

1. A casein kinase 1 (CK1) inhibitor for use in the prevention and/or treatment of vascular and/or cardiovascular diseases.

2. The CK1 inhibitor for use according to claim 1 wherein the vascular disease or cardiovascular disease is selected from the group consisting of coronary artery diseases (CAD), stroke, heart failure, hypertensive heart disease, rheumatic heart disease, cardiomyopathy, abnormal heart rhythms, congenital heart disease, valvular heart disease, carditis, aortic aneurysms, peripheral artery disease, thromboembolic disease, venous thrombosis, subarachnoid haemorrhage and hypertension.

3. The CK1 inhibitor for use according to claim 2, wherein the vascular disease or cardiovascular disease is selected from the group consisting of heart failure, subarachnoid haemorrhage and hypertension.

4. The CK1 inhibitor for use according to claim 2 wherein the CK1 inhibitor is selective for CK1δ and/or CK1ε.

5. The CK1 inhibitor for use according to claim 4 wherein the CK1 inhibitor is selected from the group consisting of PF-670462, PF-4800567 and a mixture thereof.

6. The CK1 inhibitor for use according to claim 5 wherein the CK1 inhibitor is PF-670462.

7. The CK1 inhibitor for use according to claim 5 wherein the CK1 inhibitor is PF-4800567.

8. The CK1 inhibitor for use according to claim 2 wherein the CK1 inhibitor is administered to a patient once, twice or thrice daily.

9. The CK1 inhibitor for use of claim 8 wherein the CK1 inhibitor is administered to a subject according to a regime that provides for a maximal pharmacological effect of the at least one CK1 during the resting state of the subject.

10. The CK1 inhibitor for use of claim 9 wherein the CK1 inhibitor is administered to a subject according to a regime that provides for a maximal pharmacological effect of the at least one CK1 at mid rest phase or from −2 to +2 hours of mid rest phase, preferably from −1 hours to +1 hour of mid rest phase.

11. A method for the prevention and/or treatment of a vascular and/or cardiovascular disease by administering comprising the step of administering an effective amount of at least one CK1 inhibitor to a patient in need thereof.

12. The method of claim 11 wherein the vascular disease or cardiovascular disease is selected from the group consisting of coronary artery diseases (CAD), stroke, heart failure, hypertensive heart disease, rheumatic heart disease, cardiomyopathy, abnormal heart rhythms, congenital heart disease, valvular heart disease, carditis, aortic aneurysms, peripheral artery disease, thromboembolic disease, venous thrombosis, subarachnoid haemorrhage and hypertension.

13. The method of claim 12 wherein the vascular disease or cardiovascular disease is selected from the group consisting of heart failure, subarachnoid haemorrhage and hypertension.

14. The method of claim 11 wherein the CK1 inhibitor is selective for CK1δ and/or CK1ε.

15. The method of claim 14 wherein the CK1 inhibitor is selected from the group consisting of PF-670462, PF-4800567 and a mixture thereof.

16. The method of claim 15 wherein the CK1 inhibitor is PF-670462.

17. The method of claim 15 wherein the CK1 inhibitor is PF-4800567.

18. The method of claim 11 wherein the CK1 inhibitor is administered to the patient once, twice or thrice daily.

19. The method of claim 18 wherein the CK1 inhibitor is administered to the subject according to a regime that provides for a maximal pharmacological effect of the at least one CK1 during the resting state of the patient.

20. The method of claim 19 wherein the CK1 inhibitor is administered to the patient according to a regime that provides for a maximal pharmacological effect of the at least one CK1 at mid rest phase or from −2 to about +2 hours of mid rest phase.

21. The method of claim 19 wherein the CK1 inhibitor is administered to the patient according to a regime that provides for a maximal pharmacological effect of the at least one CK1 from about −1 to about +1 hour of mid rest phase.

22. The method of claim 11 wherein the patient is a mammal, preferably a human.
